(19) United States
(12) Patent Application Publication
(10) Pub. No.: US 2006/0122112 A1
(43) Pub. Date: Jun. 8, 2006

(54) Method for preventing or treating osteosarcoma

(76) Inventors: Sho-ichi Yamagishi, Kurume (JP); Tsutomu Imaizumi, Fukuoka (JP)

Correspondence Address: WENDEROTH, LIND & PONACK, L.L.P., 2033 K STREET N. W., SUITE 800, WASHINGTON, DC, 20006-1021, US

(21) Appl. No.: 11/201,147

(22) Filed: Aug. 11, 2005

(30) Foreign Application Priority Data
Dec. 8, 2004 (JP) 2004-355711

(51) Int. Cl. A61K 38/18; A61K 48/00

(52) U.S. Cl. 514/12; 514/44

(57) ABSTRACT

Provided is a novel method for preventing or treating osteosarcoma, which comprises administering an effective amount of at least one selected from the group consisting of: (a) a pigment epithelium-derived factor; (b) a variant of the pigment epithelium-derived factor (a) that has the functionally equivalent property to the factor (a), and (c) a vector that comprises the nucleic acid molecule encoding at least one selected from the group consisting of the factor (a) and the variant (b) to a mammalian subject, especially to a human subject, in need thereof.

### TECHNICAL FIELD

The invention relates to a method for preventing or treating osteosarcoma.

### CROSS-REFERENCE TO RELATED APPLICATIONS

The present application claims the benefits of Japanese patent application number 2004-355711, filed in Japan on Dec. 18, 2004, the subject matter of which is incorporated herein by reference.

### BACKGROUND OF THE INVENTION

A major event in tumor growth and expansion is the “angiogenic switch”, an alteration in the balance of pro-angiogenic and anti-angiogenic molecules that leads to tumor neovascularization (1). Many tumors not only overexpress multiple angiogenic factors such as human vascular endothelial growth factor (VEGF), basic fibroblast growth factor and interleukin-8, but also underexpress angiogenic inhibitors such as thrombospondin-1, thus favoring angiogenesis (11, 12).

Angiogenesis, a process by which new vascular networks are formed from pre-existing capillaries, is required for tumors to grow, invade and metastasize (1, 2). Tumors are unable to grow beyond a volume of 1-2 mm³ without establishing a vascular supply because cells must be within 100-200 μm of a blood vessel to survive (1, 2). Tumor vessels are genetically stable, and less likely to accumulate mutations that allow them to develop drug resistance in a rapid manner (3). Therefore, targeting vasculatures that support tumor growth, rather than cancer cells, is considered one of the most promising approaches to cancer therapy.

Pigment epithelium-derived factor (PEDF), a glycoprotein that belongs to the superfamily of serine protease inhibitors, was first purified from human retinal pigment epithelial cell conditioned media as a factor with potent human retinoblastoma cell neuronal differentiating activity (4). Recently, PEDF has been shown to be a potent inhibitor of angiogenesis in both cell culture and animal models. Indeed, PEDF is reported to inhibit retinal endothelial cell growth, migration and suppress ischemia induced retinal neovascularization (5, 6). Furthermore, loss of PEDF was associated with angiogenic activity in proliferative diabetic retinopathy (7). PEDF is also known to effectively suppress retinal and choroidal neovascularization caused by ischemia and age-related macular degeneration, respectively (6, 13). In WO9324529, there is merely disclosed that PEDF may be useful to treat retinoblastoma, but any concrete pharmacological data to prove such activity were not taught. Very recently, Doll et al reported that PEDF-deficient mice likely suffer from pancreas cancers (14). However, a functional role of PEDF in osteosarcoma remains to be elucidated.

### SUMMARY OF THE INVENTION

An object of the present invention is to provide a novel method for preventing or treating osteosarcoma.

The present inventors have investigated in vitro growth characteristics of the human osteosarcoma cell line MG63 treated with human PEDF, and found that human PEDF exhibited a potent inhibitory effect on proliferation of osteosarcoma. Furthermore, human PEDF inhibited the expression of VEGF in human osteosarcoma cells.

The present invention relates to a method for preventing or treating osteosarcoma (including the recurrence), which comprises administering an effective amount of at least one selected from the group consisting of:

(a) a pigment epithelium-derived factor;

(b) a variant of the pigment epithelium-derived factor (a) that has the functionally equivalent property to the factor (a), and

(c) a vector that comprises the nucleic acid molecule encoding at least one selected from the group consisting of the factor (a) and the variant (b)

to a mammalian subject in need thereof. The mammalian subject may be a human subject.

### BRIEF DESCRIPTION OF THE DRAWINGS

FIG. 1 is a graph showing the dose-dependent effect of PEDF protein on growth of human MG63 osteosarcoma cells. In the figure, Ab means anti-PEDF antibody. *, P<0.05 compared with control cells without the addition of PEDF.

FIG. 2 is a graph showing the inhibition of the expression of VEGF in human MG63 osteosarcoma cells by PEDF.

### DETAILED DESCRIPTION OF THE PREFERRED EMBODIMENTS

1. Proteins

In the present invention, “pigment epithelium derived factor” or “PEDF” means pigment epithelium-derived factor (PEDF) protein. The amino acid sequence and the nucleic acid sequence of PEDF are described in Proc. Natl. Acad. Sci. U.S.A. 90 (4), 1526-1530 (1993) (this reference is incorporated herein by reference), and are registered under Genbank Accession No. M76979. For reference, the amino acid sequence and the nucleic acid sequence of PEDF are shown in SEQ ID Nos.: 1 and 2, respectively. According to the invention, PEDF includes all kinds of PEDF derived from mammals such as human, dog, cat, cow, horse and monkey. When the subject to be treated by the invention is human, they should have the functionally equivalent property to human PEDF and PEDF derived from human is preferred.

Production of PEDF by expressing the DNA encoding the protein may be achieved in accordance with many publications and references such as “Molecular Cloning”, 2nd ed., Cold Spring Harbor Laboratory Press (1989). Particularly, an expression plasmid is constructed by inserting a DNA of the present invention into an appropriate expression vector (e.g., pBK-CMV). Subsequently, the expression plasmid is introduced into appropriate host cells to obtain transformants. Examples of host cells include those cells of prokaryotes such as Escherichia coli, unicellular eukaryotes such as yeast, and multicellular eukaryotes such as insects or animals.

Transfer of expression plasmid into host cells may be achieved by conventional methods such as calcium phosphate method, electric pulse method, Lipofection method, or the like. Desired proteins are produced by culturing the transformants in appropriate medium according to conventional methods. The protein thus obtained may be isolated and purified according to standard biochemical procedures.

As used herein, “variant of PEDF that has the functionally equivalent property to the PEDF” includes all kinds of PEDF variants as long as the variants have the functionally equivalent property to the PEDF. For example, the variants of PEDF are described in, for example, U.S. Pat. No. 6,319,687, WO03/059248 and WO93/24529 (those references are incorporated herein by reference).

Preferable examples of the PEDF variants include variants of PEDF that comprise an amino acid sequence that contains alteration of one or more, or several amino acid residues in the amino acid sequence of human PEDF wherein the alteration is substitution, deletion and/or addition, and have the functionally equivalent property to human PEDF.

“The functionally equivalent property to human PEDF” means the property to inhibit cell proliferation and/or VEGF expression in human osteosarcoma cells.

The variants according to the invention may also be prepared by recombinant technology as shown above.

It can be demonstrated whether or not a variant prepared as shown above has the functionally equivalent property according to the examples hereinafter.

According to the method of the present invention, PEDF or a variant thereof can be administered, if necessary, in a form of a pharmaceutical composition with a conventional carrier.

According to the method of the present invention, the patient may be any mammal such as human, dog, cat, cow, horse or monkey and preferably, human patient.

According to the present invention, a PEDF or a variant thereof may be administered in such a manner that they contact with osteosarcoma, for example, intradermally, hypodermically, or by intravenous injection. Preferably, a PEDF or a variant thereof is administered by injection to the site where the osteosarcoma exist directly, or by use of an antibody directed to the osteosarcoma. Any conventional method may be used to target osteosarcoma. The amount of the protein to be administered may vary depending on the severity of the condition to be treated, the age and the weight of the patient, and the like. For an adult male patient (body weight about 60 kg), it is typical to administer 0.0001 mg-1000 mg, preferably 0.001 mg-100 mg, more preferably 0.01 mg-10 mg of a PEDF or a variant thereof every several days to every several months.

2. Nucleic Acids and Vectors

The present invention also provides a method for preventing or treating osteosarcoma which comprising administering a vector that comprises a nucleic acid that encodes a PEDF, or a variant thereof that comprises an amino acid sequence that contains alteration of one or several amino acid residues in the amino acid sequence of the PEDF wherein the alteration is substitution, deletion and/or addition, and has the functionally equivalent property to the PEDF.

As used herein, “nucleic acid” includes a DNA and an RNA, which may be single-stranded or double-stranded. Nucleic acid can be easily prepared according to typical DNA synthesis or genetic engineering method, for example, according to the description of a standard text such as “Molecular Cloning”, 2nd ed., Cold Spring Harbor Laboratory Press (1989).

According to the present invention, the vector should be designed so that the nucleic acid encoding a PEDF or a variant thereof incorporated in the vector can be highly expressed in the osteosarcoma cells.

Such vectors mean those useful for gene therapy as used conventionally. Examples of the vectors include viral vectors wherein the nucleic acid as shown above is incorporated into DNA or RNA viruses such as retrovirus, adenovirus, adeno-associated virus, herpes virus, vaccinia virus, poxvirus, poliovirus, or Sindbis virus, and introduced into cells. Among these methods, those using retrovirus, adenovirus, adeno-associated virus, or vaccinia virus are especially preferred. Specific examples of the vectors include pAd/CMV/V5-DEST Gateway Vector (Invitrogen).

According to the present invention, a pharmaceutical composition comprising such a vector of the present invention as an active ingredient and, if necessary, conventional carriers may be used.

According to the present invention, the vector may be administered, for example, intradermally, hypodermically, or by intravenous injection. Preferably, the vector is administered by injections to the site where the osteosarcoma masses exist directly, or by use of antibodies directed to the osteosarcoma. Any conventional method may be used to target osteosarcoma. Although the amount of the vector to be administered may vary depending on the severity of the condition to be treated, the age and the weight of the patient and the like, it is typical to administer 0.0001 mg-100 mg, preferably 0.001 mg-10 mg, of a nucleic acid every several days to every several months.

### EXAMPLES

The present invention is further illustrated by the following examples, but is not limited by these examples in any respect.

All values were represented as means±S. E. M. (standard error of the mean). Statistical significance was evaluated using the Student's t test for paired comparison; p<0.05 was considered significant.

1. Preparation of PEDF-Expression Vectors

PEDF cDNA was originally cloned from a human placenta cDNA library (Clontech, Palo Alto, Calif.), and inserted into the mammalian expression vector pBK-CMV (Stratagene, La Jolla, Calif.) as described in Reference 8.

In brief, the gene encoding PEDF was isolated from the cDNA library by PCR according to the following conditions:

PCR Primer

Fw: 5′-CTCAGTGTGCAGGCTTAGAG-3′(SEQ ID NO: 4)
Rev: 5′-CCTTCGTGTCCTGTGGAATC-3′(SEQ ID NO: 5)

| | | |
|---|---|---|
| ×10 pfu buffer | 4 | μl |
| dNTP (2 mM each) | 4 | μl |
| primer (Fw) (10 μM) | 4 | μl |
| primer (Rev) (10 μM) | 4 | μl |
| templates (200 mg) | | |
| pfu polymerase (STRATAGENE) | 0.5 | μl |
| distilled H₂O | /total 40 | μl |

Condition of PCR (Parkin Elmer 2400)

(step 1 × 1)

| | |
|---|---|
| 95° C. | 5 min. |

(step 2 × 30)

| | |
|---|---|
| 95° C. | 0.5 min. |
| 60° C. | 0.5 min. |
| 72° C. | 1.5 min. |

(step 3 × 1)

| | |
|---|---|
| 72° C. | 10 min. |
| 4° C. | ∞ min. |

After confirmation of the amplification, the PCR product was ligated to the SmaI site of pBluescript II KS. The construction was confirmed by digestion with restriction enzymes and sequencing, and also checked to contain the Xba I site at the 5′ side.

The PEDF PCR product that had been cut with Xba I and Hind III (bulnted) was cloned into pBK-CMV (STRATAGENE) between Nhe I (blunted) and Xba I sites, to give a PEDF expression vector, plasmid pBK-CMV-PEDF.

Then, an expression vector for purification of PEDF was constructed according to the following procedure.

OligoDNAs for His-Tag, 5′-AATTCCATCATCATCATCATCATTAAT-3′ (SEQ ID NO: 6) and 5′-CTAGATTAATGATGATGATGATGATGG-3′ (SEQ ID NO: 7) were synthesized, and annealed each other. To eliminate the stop codon of PEDF and insert an Eco RI site at the 3′ end, an oligoDNA was synthesized by PCR using 5′-CGGAATTCGGGGCCCCTGGGGTCC-3′ (SEQ ID NO: 8) and the Fw primer (SEQ ID NO: 4) described above, and using pBK-CMV-PEDF as template. The amplified product was cut with Bgl II and Eco RU, and the cut product was ligated to the annealed oligoDNA for His Tag as described above. The ligated product was inserted into the purified pBK-CMV-PEDF which had been cut with Bgl II and Xba I. The construction was confirmed by digestion with restriction enzymes and sequencing.

To insert a translational enhancer, the enhancer segment which was isolated from pcDNA4-HisMax (Invitrogen) using Sac I and Xba I, was ligated to the same restriction enzyme sites of pBluescript II KS. After isolation of the clone, the vector was cut at the Nco I site (blunted) and Bam HI site to give the vector backbone.

To modify the 5′ end of PEDF, an oligoDNA was synthesized using 5′-GCATGCAGGCCCTGGTGCTACTCC-3′ (SEQ ID NO: 9) (Sph I site was inserted to the PEDF 5′ end) and 5′-TTAGGTACCATGGATGTCTGGGCT-3′ (SEQ ID NO: 10), and using pBK-CMV-PEDF as template, and the synthesized product was inserted into pGEM-T easy (Promega). The clone was isolated, and then cut at Sph I site (blunted) and Bam HI site. The resulting fragment was inserted into the vector backbone described above.

The translational enhancer-PEDF 5′ portion was removed from the resulting vector by cutting its Sac I site (blunted) and Bam HI site. The fragment containing the translational enhancer-PEDF 5′ portion was ligated to pBK-CMV-PEDF having 3′ His-Tag which had been cut at the Nhe I site (blunted) and Bam HI site to give an expression vector for purification of PEDF. The resulting vector was confirmed by digestion with restriction enzyme and sequencing.

2. Preparation of PEDF Proteins

293T cells (ATCC No. CRL-11268, ATCC, Rockville, Md.) were transfected with an expression vector for purification of PEDF as prepared above using the FuGENE 6 transfection reagent (Roche Diagnostics, Mannheim, Germany) according to the manufacturer's instructions. Then, PEDF proteins were purified from conditioned media by a Ni-NTA spin kit (Qiagen GmbH, Hilden, Germany) according to the manufacture's instructions. SDS-PAGE analysis of purified PEDF proteins revealed a single band with a molecular weight of about 50 kDa, which showed reactivity with the previously described Ab against human PEDF (8).

3. Preparation of Polyclonal Antibodies (Ab) Against Human PEDF

Polyclonal Ab against 44-mer PEDF polypeptides (VLLSPLSVATALSALSLGAEQRTESIIHRALYYDLISSFDIHGT: SEQ ID NO: 3) was prepared as previously described (8). The present inventors confirmed that the polyclonal Ab actually bound to purified PEDF protein.

4. Effects on Osteosarcoma Cells

4.1 Inhibition of Cell Proliferation

MG63 human osteosarcoma cells, which were available from Riken Cell Bank, were cultured in Dulbecco's modified Eagle's medium (DMEM) supplemented with 10% of fetal bovine serum (FBS) (ICN Biomedicals Inc., Aurora, Ohio, USA) and 100 units/ml penicillin/streptomycin. To the medium, 1 nM, 10 nM or 100 nM of PEDF, which had been prepared as above, was added, and cultured for 2 days, and the viable cell numbers were countered according to the method described by S. Yamagishi, et al., Journal of Biological Chemistry, 1997, 272 (13), 8723-8730 (this reference is incorporated herein by reference).

For the comparison, the similar experiments were conducted either by adding the known anti-tumor agent, methotrexate, in place of PEDF or without adding PEDF.

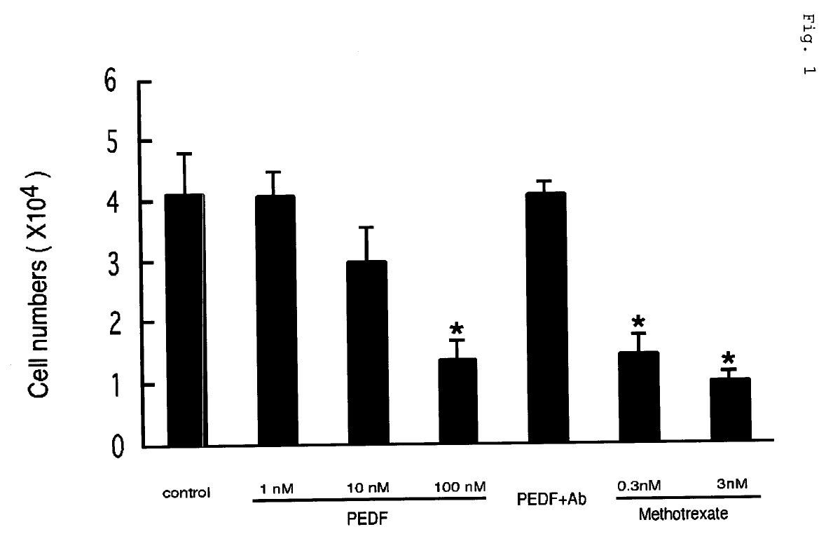

As shown in FIG. 1, PEDF dose-dependently decreased the viable cell numbers. PEDF at the concentration of 100 nM decreased the cell numbers to less than 50% of that in the control. This effect is almost equivalent to that of 0.3 nM of methotrexate. This effect of PEDF was disappeared by adding anti-PEDF antibody prepared as above. Those results demonstrate that PEDF directly affects osteosarcoma cells and inhibits the cell proliferation.

4.2 Decreased Expression of VEGF mRNA

VEGF is one of the most important growth factors involved in angiogenic switch in human tumors. PEDF acted against MG63 osteosarcoma cells, and inhibited the expression of VEGF which plays the most important role for the induction of the angiogenesis in tumor cells.

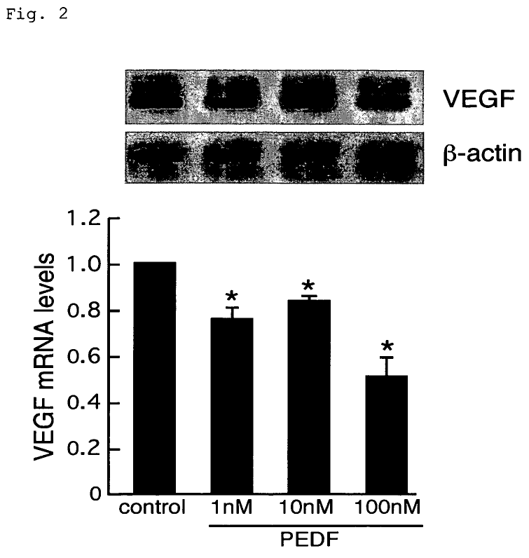

Namely, Poly(A)⁺RNAs were isolated from MG63 cells treated with or without the indicated concentrations of PEDF for 4 hours, and then the amount of human VEGF mRNA was analyzed by RT-PCR. The primer for the detection of human VEGF mRNA was described, for example, by S. Yamagishi et al., Journal of Biological Chemistry, 2002, 277(23), 20309-20315 (this reference is incorporated herein by reference). RT-PCR method was reported by M. Nomura et al., Journal of Biological Chemistry, 1995, 270(47), 28316-28324 (this reference is incorporated herein by reference). As summarized in FIG. 2, the results of the measurement shows that the 100 nM PEDF treatment group decreased to almost 50% level of the expression of human VEGF mRNA as compared the non-treatment group. This result indicates that PEDF inhibits secretion of VEGF from osteosarcoma cells, leading to inhibition of tumor angiogenesis.

### REFERENCES

The contents of the references cited in the specification and below are herein incorporated by reference.
1. Holmgren, L., O'Reilly, M. S., and Folkman, J. Dormancy of micrometastases: balanced proliferation and apoptosis in the presence of angiogenesis suppression. Nat Med. 1:149-53. 1995.
2. Carmeliet, P., and Jain, R. K. Angiogenesis in cancer and other diseases. Nature. 407:249-257. 2000.
3. Scappaticci, F. A. Mechanisms and future directions for angiogenesis-based cancer therapies. J Clin Oncol. 20:3906-3927. 2002.
4. Tombran-Tink, J., Chader, C. G., and Johnson, L. V. PEDF: a pigment epithelium-derived factor with potent neuronal differentiative activity Exp Eye Res. 53:411-414. 1991.
5. Dawson, D. W., Volpert, O. V., Gillis, P., Crawford, S. E., Xu, H. J., Benedict, W., and Bouck, N. P. Pigment epithelium-derived factor: a potent inhibitor of angiogenesis. Science. 285:245-248. 1999.
6. Duh, E. J., Yang, H. S., Suzuma, I., Miyagi, M., Youngman, E., Mori, K., Katai, M., Yan, L., Suzuma, K., West, K., Davarya, S., Tong, P., Gehlbach, P., Pearlman, J., Crabb, J. W., Aiello, L. P., Campochiaro, P. A., and Zack, D. J. Pigment epithelium-derived factor suppresses ischemia-induced retinal neovascularization and VEGF-induced migration and growth. Invest Ophthalmol Vis Sci. 43:821-829, 2002.
7. Spranger, J., Osterhoff, M., Reimann, M., Mohlig, M., Ristow, M., Francis, M. K., Cristofalo, V., Hammes, H. P., Smith, G., Boulton, M., and Pfeiffer, A. F. Loss of the antiangiogenic pigment epithelium-derived factor in patients with angiogenic eye disease. Diabetes. 50:2641-26415. 2001.
8. Yamagishi, S., Inagaki, Y., Amano, S., Okamoto, T., Takeuchi, M., and Makita, Z. Pigment epithelium-derived factor protects cultured retinal pericytes from advanced glycation end product-induced injury through its antioxidative properties. Biochem Biophys Res Commun. 296:877-882. 2002.
9. Rofstad, E. K., and Halsoer, E. F. Vascular endothelial growth factor, interleukin 8, platelet-derived endothelial cell growth factor, and basic fibroblast growth factor promote angiogenesis and metastasis in human melanoma xenografts. Cancer Res. 60:4932-4938. 2000.
10. Reiher, F. K., Volpert, O. V., Jimenez, B., Crawford, S. E., Dinney, C. P., Henkin, J., Haviv, F., Bouck, N. P., and Campbell, S. C. Inhibition of tumor growth by systemic treatment with thrombospondin-1 peptide mimetics. Int J Cancer. 98:682-689. 2002.
11. Holekamp, N. M., Bouck, N., and Volpert, O. Pigment epithelium-derived factor is deficient in the vitreous of patients with choroidal neovascularization due to age-related macular degeneration. Am J Ophthalmol. 134:220227. 2002.
12. Doll, J. A., Stellmach, V. M., Bouck, N. P., Bergh, A. R., Lee, C., Abramson, L. P., Cornwell, M. L., Pins, M. R., Borensztajn, J., and Crawford, S. E. Pigment epithelium-derived factor regulates the vasculature and mass of the prostate and pancreas. Nat Med. 9:774-80. 2003.
13. Holekamp, N. M., Bouck, N., and Volpert, O. Pigment epithelium-derived factor is deficient in the vitreous of patients with choroidal neovascularization due to age-related macular degeneration. Am J Ophthalmol. 134:220227. 2002.
14. Doll, J. A., Stellmach, V. M., Bouck, N. P., Bergh, A. R., Lee, C., Abramson, L. P., Cornwell, M. L., Pins, M. R., Borensztajn, J., and Crawford, S. E. Pigment epithelium-derived factor regulates the vasculature and mass of the prostate and pancreas. Nat Med. 9:774-80. 2003.
15. Abe R, Shimizu T, Yamagishi S, Shibaki A, Amano S, Inagaki Y, Watanabe H, Sugawara H, Nakamura H, Takeuchi M, Imaizumi T, Shimizu H. Overexpression of pigment epithelium-derived factor decreases angiogenesis and inhibits the growth of human malignant melanoma cells in vivo. Am J Pathol. 2004 April;164(4):1225-32.
16. Takenaka K, Yamagishi S I, Jinnouchi Y, Nakamura K, Matsui T, Imaizumi T. Pigment epithelium-derived factor (PEDF)-induced apoptosis and inhibition of vascular endothelial growth factor (VEGF) expression in MG63 human osteosarcoma cells. Life Sci. 2005 Jun. 25; [Epub ahead of print].

What is claimed is:

1. A method for preventing or treating osteosarcoma, which comprises administering an effective amount of at least one selected from the group consisting of:
(a) a pigment epithelium-derived factor;
(b) a variant of the pigment epithelium-derived factor (a) that has the functionally equivalent property to the factor (a), and
(c) a vector that comprises the nucleic acid molecule encoding at least one selected from the group consisting of the factor (a) and the variant (b) to a mammalian subject in need thereof.

2. The method of claim 1, wherein the mammalian subject is a human subject.

3. The method of claim 2, wherein the pigment epithelium-derived factor is that derived from human.


SEQUENCE LISTING (10 sequences)

SEQ ID NO: 1 - protein, 418 aa, homo sapiens [sequence omitted]

SEQ ID NO: 2 - DNA, 1490 bp, homo sapiens
ggacgctggattagaaggcagcaaaaaaagatctgtgctggctggagccccctcagtgtgcaggcttagagggactaggctgggtgtggagctgcagcgtatccacaggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcatgagccccacgaccaacgtgctcctgtctcctctcagtgtggccacggccctctcggccctctcgctgggagcggacgagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctrcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttacgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccct

SEQ ID NO: 3 - protein, 44 aa, Artificial Sequence
  PEDF-derived peptide
VLLSPLSVATALSALSLGAEQRTESIIHRALYYDLISSFDIHGT

SEQ ID NO: 4 - DNA, 20 bp, Artificial Sequence
  PCR primer
ctcagtgtgcaggcttagag

SEQ ID NO: 5 - DNA, 20 bp, Artificial Sequence
  PCR primer
ccttcgtgtcctgtggaatc

SEQ ID NO: 6 - DNA, 27 bp, Artificial Sequence
  OligoDNA for His Tag
aattccatcatcatcatcatcattaat

SEQ ID NO: 7 - DNA, 27 bp, Artificial Sequence
  OligoDNA for His Tag
ctagattaatgatgatgatgatgatgg

SEQ ID NO: 8 - DNA, 24 bp, Artificial Sequence
  PCR primer
cggaattcggggcccctggggtcc

SEQ ID NO: 9 - DNA, 24 bp, Artificial Sequence
  PCR primer
gcatgcaggccctggtgctactcc

SEQ ID NO: 10 - DNA, 24 bp, Artificial Sequence
  PCR primer
ttaggtaccatggatgtctgggct